(19) United States
(12) Patent Application Publication
(10) Pub. No.: US 2010/0324455 A1
(43) Pub. Date: Dec. 23, 2010

(54) DEVICES FOR MANAGEMENT OF FOOT INJURIES AND METHODS OF USE AND MANUFACTURE THEREOF

(75) Inventors: Paulo RANGEL, Carlsbad, CA (US); Charles G. SIPES, JR., Harrisville, RI (US); Michael A. WHITTAKER, San Diego, CA (US); Craig Steven GUNTHER, Mission Viejo, CA (US)

Correspondence Address: BioTechnology Law Group;12707 High Bluff Drive, Suite 200, San Diego, CA, 92130-2037, US

(73) Assignee: LASERCURE SCIENCES, INC., Carlsbad, CA (US)

(21) Appl. No.: 12/785,446

(22) Filed: May 22, 2010

Related U.S. Application Data
(60) Provisional application No. 61/180,849, filed on May 23, 2009.

(51) Int. Cl. A61B 5/103; A61F 5/00; A43B 13/38

(52) U.S. Cl. 600/592; 602/28; 36/43

(57) ABSTRACT

The present invention provides orthotic devices for use in managing the treatment and prevention of lower extremity injuries, including foot ulcers. In various aspects, the present invention provides foot-worn orthotics which provide for improved compliance monitoring, and methods of their manufacture and use.

### CROSS REFERENCE TO RELATED APPLICATIONS

The present invention claims priority from U.S. Provisional Patent Application 61/180,849 filed May 23, 2009, which is hereby incorporated by reference in its entirety including all tables, figures and claims.

### BACKGROUND OF THE INVENTION

The following discussion of the background of the invention is merely provided to aid the reader in understanding the invention and is not admitted to describe or constitute prior art to the present invention.

Many of the estimated 20 million individuals in the United States with diabetes mellitus (“DM”) will experience pathologic changes of their lower extremities that, when combined with minor trauma and infection, may lead to serious foot problems. The most common dermatologic manifestations of DM are poor healing of wounds and skin ulceration. Approximately 15% of DM sufferers will develop at least one foot ulcer, and a substantial percentage of these (14-24%) will require amputation. Risk factors for foot ulceration include diabetes for more than 10 years, poor blood sugar control, peripheral artery disease, smoking, history of previous ulcers, male sex, and presence of calluses.

The underlying reasons for the incidence of lower extremity ulcers in DM is a combination of peripheral neuropathy, peripheral arterial disease, and poor wound healing, each of which is increased DM. These combine to alter the normal anatomy of the foot in many cases, and interfere with normal protective mechanisms due to reduced sensory input. The altered blood flow leads to drying of the skin surface, which can cause fissures to form. These breaks in the skin enlarge and often become infected.

In addition to diabetic ulcers, lower extremity ulcers may also be caused by venous stasis and arterial ischemia. Venous ulcers are located below the knee and are primarily found on the inner part of the leg, just above the ankle. Venous stasis ulcers are common in patients who have a history of leg swelling, long standing varicose veins, or a history of blood clots in either the superficial or the deep veins of the legs. Arterial ulcers are usually located on the feet and often occur on the heels, tips of toes, between the toes where the toes rub against one another or anywhere the bones may protrude and rub against bed sheets, socks or shoes. Arterial ulcers also occur commonly in the nail bed if the toenail cuts into the skin or if the patient has had recent aggressive toe nail trimming or an ingrown toenail removed.

Management of lower extremity ulcers is largely determined by severity, vascularity, and the presence of infection. All necrotic, callus, and fibrous tissue is often debrided back to bleeding tissue to allow full visualization of the extent of the ulcer and detect underlying abscesses or sinuses. A warm, moist environment that is protected from external contamination can be provided by a number of commercially available special dressings, including semipermeable films, foams, hydrocolloids, and calcium alginate swabs. Platelet-derived growth factor (Regranex gel) is approved for use on neuropathic diabetic foot ulcers and can expedite healing. Bioengineered skin (Apligraf) and human dermis (Dermagraft) are new types of biologically active implants for ulcers that are derived from human fibroblasts. When infection is present, aerobic and anaerobic cultures should be obtained, followed by initiation of appropriate broad-spectrum antibiotic therapy. Surgical drainage, deep debridement, or local partial foot amputations are necessary adjuncts to antibiotic therapy of infections that are deep or limb-threatening. In addition, low level light therapy (LLLT) in the near-IR and/or IR wavelengths has been used to stimulate local nitric oxide production for treatment of a variety of ulcers, including lower extremity ulcers. See, e.g., U.S. Pat. Nos. 6,454,791 and 6,156,028, and WO06/113269.

Rest, elevation of the affected foot, and relief of pressure are essential components of treatment and should be initiated at first presentation. Ill-fitting footwear should be replaced with a postoperative shoe or another type of pressure-relieving footwear, or by total off-loading of pressure from the foot. As gait velocity has an effect on plantar pressure distribution, mainly in the toes and heel region, patients may be instructed to walk slowly in order to protect the foot from high peak pressures. In addition, patients reportedly wear off-loading devices for only a minority of steps taken each day. This has led to the use of cast boots, which force compliance with off-loading as the boot is not removable by the patient, but which also prevent dressing changes, cleaning, and monitoring of the foot without removing the cast.

### SUMMARY OF THE INVENTION

The present invention provides orthotic devices for use in managing the treatment and prevention of lower extremity injuries, including foot ulcers, and soft tissue injuries including plantar fasciitis. In various aspects, the present invention provides foot-worn orthotics which enable and monitor user compliance, and methods of their manufacture and use. Such devices can also act as “virtual casts” which monitor mobility of the lower extremity and can notify the user or caregiver when that mobility exceeds predetermined mobility parameters. Thus, the present invention can be used in the same way that traditional casts, braces, wraps, etc., are used by caregivers to limit mobility of (“to immobilize”) an extremity to aid the healing process, and as an alternative or substitute for diabetic or other patients who may have lost sensation in certain areas and require a proxy to mobility parameters in order to avoid harming themselves or impairing treatment.

In a first aspect of the invention, the present invention provides orthotics for monitoring delivery of therapy to an injured foot. The footwear orthotics of the present invention senses and monitors periods of placement of the orthotic on the foot as well as periods of weight-bearing use, and stores data related to this monitoring for later download and analysis by a caregiver. These footwear orthotics comprise:
(i) a sensor array containing at least one proximity sensor configured to generate an electronic signal indicative of placement of the orthotic on a foot of a wearer;
(ii) a computer processor operably connected to said sensor array, wherein said computer processor receives data indicative of periods of placement of the orthotic on said foot stores said data for future retrieval;
(iv) a power supply operably connected to said at least one proximity sensor, said at least one pressure sensor, and said computer processor; and
(v) a communications circuit configured to provide communication of data generated by said sensor array, or a processed form thereof, to a database, display, or to a second computer processor external to said orthotic.

In certain embodiments, the footwear orthotics of the present invention monitor periods of placement of the orthotic on the foot as well as periods of weight-bearing use. In these embodiments, the footwear orthotics comprise:
(i) a sensor array containing at least one proximity sensor configured to generate an electronic signal indicative of placement of the orthotic on a foot of a wearer, and at least one pressure sensor configured to generate an electronic signal indicative of weight-bearing use of the orthotic by said wearer;
(iii) a computer processor operably connected to said sensor array, wherein said computer processor receives data indicative of periods of placement of the orthotic on said foot and periods of weight bearing use of the orthotic by said wearer and stores said data for future retrieval;
(iv) a power supply operably connected to said at least one proximity sensor, said at least one pressure sensor, and said computer processor; and
(v) a communications circuit configured to provide communication of data generated by by said sensor array, or a processed form thereof, to a database, or to a second computer processor external to said orthotic.

In certain embodiments, the pressure sensor(s) comprise(s) a plurality of independent pressure sensor locations which sense pressure at a plurality of locations on the orthotic. Because of the variations in foot anatomy from individual to individual, the profile of pressure signals obtained from the plurality of locations can act as a biometric signature of the wearer. This can provide compliance information (e.g., preventing the user from circumventing a proximity sensor), and can serve as a signal to the device to initiate a treatment protocol.

In an alternative, or together with such pressure sensor(s), the orthotic of the present invention may be coupled to a user input device (e.g., a keypad, touchscreen, accelerometer, fingerprint reader, etc.) which is triggered by an affirmative action by the user (e.g., entry of a code or a biometric key). This trigger, together with a signal from the proximity sensor, can serve as a signal to the device to initiate a treatment protocol.

The term “footwear orthotic” as used herein refers to any device adapted for wear on the foot. This term includes shoes, boots, sandals, socks, etc., as well as insoles which are separable elements configured for placement within another footwear orthotic. Preferably, the footwear orthotics of the present invention are adapted for repeated cycles of attachment and removal from the foot, as in the case of a conventional shoe or boot which comprises hook and loop straps, pull ties, laces, zippers etc., for reversible closure or tightening, or a sock, sandal, or slip-on shoe which may be worn without closure or tightening of a fastener. This list is not meant to be limiting. Placement of such an orthotic on the foot is not meant to indicate that the foot is otherwise uncovered; the foot may also be covered by a sock or wound dressing for example.

The term “proximity sensor” as used herein refers to a sensor able to detect the presence of nearby objects and generate an electronic signal in response. Types of proximity sensors known in the art include capacitive, magnetic, optical (e.g., reflective or passive), ultrasonic, thermal (e.g., infrared), etc. This list is not meant to be limiting. Preferred proximity sensors are calibrated to provide a positive signal if an object is within 5 mm or less of the sensor. The term “signal indicative of placement of the orthotic on a foot of a wearer” as used herein refers to an electronic signal generated by proximity sensor(s) in an orthotic which are characteristic of proper placement of the orthotic on the foot. Such signals may also be generated in error, for example by placing ones hands over the proximity sensor(s), but would still be considered to be “indicative” of placement of the orthotic on the foot if the signals are those expected to be generated by placement of the orthotic on the foot.

The term “pressure sensor” as used herein refers to a sensor able to detect changes in pressure and generate an electronic signal in response. Types of pressure sensors known in the art include mechanical (e.g., a simple spring switch which closes under an increased pressure condition and opens when the pressure is released), strain, piezoresistive, variable capacitance, etc. This list is not meant to be limiting. In certain embodiments, a pressure sensor is a distributed pressure sensing material that is able to detect and optionally record a pressure profile across a surface area of the orthotic. This can permit the orthotc to dynamically respond and to adjust to changes in weight and position of the foot with time.

The term “computer processor” refers to electronic circuits such as microprocessors, digital signal processors and microcontrollers that can execute a set of instructions. In certain embodiments the processor is operably connected to a memory which can store programming information for access and execution by the computer processor, and/or can store data from the computer processor, such as data generated by the sensors described herein. Types of memories known in the art include “flash”-type (electrically erasable programmable read-only) memory, static random access memory, dynamic random access memory, etc. Preferred memories are non-volatile memories such as flash and SRAM memories.

The term “power supply” as used herein refers to a source of electrical power. Preferred power supplies are batteries, as this type of supply offers mobility and portability to the present orthotics. Batteries can include disposable cells (e.g., zinc-carbon, zinc chloride, alkaline, silver-oxide, lithium-thionyl chloride, mercury, zinc-air, etc.) and/or rechargeable cells (e.g., nickel-cadmium, nickel hydrogen, nickel-metal hydride, lithium ion, lithium ion polymer, lithium sulfur, rechargeable alkaline, lithium iron phosphate, etc.). A power supply may contain two or more batteries; for example, a first battery may be used to power certain electronic elements within the orthotic, and a second battery used to maintain a volatile memory component.

A battery power supply may be charged in a number of fashions. For example, the orthotic may comprise a connector in electrical communication with the power supply to provide an external connection on the orthotic which mates with a corresponding connection on a battery charger, providing for a direct wired contact path with the power supply. Alternatively, the battery power supply is in electrical communication with an induction coil, and energy is provided to charge the power supply through inductive coupling to an external induction coil in a battery charger. Inductive charging provides battery contacts which can be completely sealed within the orthotic to prevent exposure of the electronics to water or other contamination.

In certain embodiments, the orthotic can be provided with regenerative charging of the battery power supply in order to extend battery life. The term “regenerative charging” as used herein refers to circuitry which converts kinetic energy of movement into electrical energy for battery storage. Such circuitry can include an electromagnetic generator (e.g., one or more magnets moving in a wire coil inducing electromagnetic power). Due to the movements of the user, the magnet(s) will swing in and out of the wire coil and induce voltage in the coils. The harvested energy from this block will be transferred to a step-up transformer (e.g., wires coiled around metal bars). The transformer will receive the output of the generator and step it up to the optimum output required for the battery. A rectifier will convert the electrical output from the transformer from AC to DC in order to charge the batteries, and a regulator to regulate the voltage supplied and ensure that the power stored in the battery does not leak back into the charging device.

The term “communications circuit” as used herein refers to circuitry providing wired or wireless communications for purposes of accessing programming and/or data storage location(s) within the orthotic. Wired communications circuits include the provision of connection ports (e.g., USB ports), a display, removable memory cards, etc., as a component of the orthotic or separate from the orthotic but in wired communication therewith (whether permanently connected or reversibly connected). In this regard, a display may include any electronic element capable of providing a visual indication of data including, but not limited to a screen capable of displaying alphanumeric characters. Wireless communications circuits include radio frequency and optical (e.g., infrared) circuitry. In certain embodiments the wireless communications circuitry provides communications by means of known communications protocols such as bluetooth, HomeRF, IEEE 802.11b, IEEE 802.11a, IEEE 802.15.4, CDMA, TDMA, GSM, and WAP. This list is not meant to be limiting.

The term “sensor array” as used herein refers to one or more sensors, including pressure, proximity, temperature, humidity, heart rate and other sensors, that are used to detect and collect data about the patient while the orthotic is worn. In certain embodiments, a sensor array can be formed in a laminated fashion, for example with a distributed pressure sensor configured to detect pressures at a plurality of locations in the orthotic in a first layer, and proximity and/or other sensor types in a second layer which may be positioned closer to the user (e.g., in a top layer overlying the pressure sensor layer).

The present invention replies on a combination of proximity sensor(s) and pressure sensor(s) to determine periods of use of the orthotic, and to create a “virtual cast” that may be combined with boots or other devices to monitor and record immobilization and orthotic use consistent with caregiver instruction, without obstruction and cost of the physical cast, and that may dynamically be reconfigured or tuned in response to the patient's needs. For example, the computer processor may be configured to store data indicative of placement of the orthotic on the foot of a wearer when the computer processor receives indicative electronic signals from a single proximity sensor in the orthotic. While proximity sensor(s) are preferably placed to be contacted by the sole portion of a wearer's foot, this need not be the case. Proximity sensors may be placed at any location in the orthotic that will be expected to come within the sensing distance of the sensor during proper use of the orthotic.

In the case of a single proximity sensor, such a sensor may be falsely triggered by putting an object into or onto the orthotic sufficiently close to trigger the sensor. Thus, in certain embodiments, the orthotics of the present invention comprise at least two proximity sensors. Preferably at least two proximity sensors are spatially separated by 5 cm or more, and most preferably 10 cm or more. By separating the sensors, the proximity sensors are less likely to be inadvertently triggered simultaneously. In such a case, the computer processor may be configured to store data indicative of placement of the orthotic on the foot of a wearer only when the computer processor receives indicative electronic signals from each of two or more proximity sensors simultaneously. In certain embodiments, the orthotic comprises at least one proximity sensor positioned in the metatarsal region and at least one proximity sensor positioned between the heel and midsole regions.

In an alternative to limit false triggers, the orthotic may rely on a combination of proximity sensors and biometric signals and/or affirmative actions by the user to signal the computer processor of proper placement on the wearer. This can improve safety of the devices by coupling initiation of treatment to receipt of the desired combination of signals; improve the caregiver's understanding of compliance by the user; and provide feedback to the user and caregiver concerning the treatment regimen being employed. A user interface can also permit the wearer to record periods of discomfort and respond to queries from the computer processor regarding error states (e.g., low battery signals, signals that use is outside expected parameters, etc.

Pressure sensor(s) may be placed under the foot of the wearer, such that standing will put force on the sensor. In certain embodiments, the pressure sensor(s) are configured to measure pressure magnitudes exerted on certain location(s) in the orthotic in order to determine the success of off-loading, periods of extreme pressure, locations of extreme pressure, etc. These pressure magnitudes may be stored by the computer processor. In certain other embodiments, the pressure sensor is configured to trigger when a certain pre-set force is exceeded. This pre-set force may be small, such that use of the orthotic by the wearer to bear a certain amount of weight will trigger the sensor. Alternatively, or in conjunction, a pre-set force may also be relatively large, such as a force which is detrimental to healing of a foot ulcer. Such a force may be at least twice the force of a force felt by the pressure sensor when the wearer stands at rest on his or her feet, at least 5 times such a force, or more. The computer processor can store periods of weight bearing use of the orthotic detected by the pressure sensor(s), periods of walking (e.g., when the pressure sensor is triggered in a repetitive fashion indicating the gait of the wearer), gait velocity (e.g., the frequency of triggering), periods of high force (e.g., when the gait of the wearer is sufficiently rapid to indicate a high gait velocity), or a combination of such events.

In another embodiment, the data generated from the pressure sensor(s) may be processed and used to generate a profile of the force vectors on the foot under normal walking, running, standing and other conditions. Variations from normal profiles for the patient that are stored in a database can be detected, and adjustments to the orthotic, walking device or treatment can be made at the onset of a problem. Alternatively, the orthotic or other walking devices such as prosthetics can be tuned and balanced using this feedback.

The data indicating placement of the orthotic on a foot of a wearer and its weight-bearing use may be accessed by an external device for various purposes. For example, a caregiver may access the stored data to determine compliance with the desired off-loading protocol and to provide guidance for future care (for example, if the patient is complying with a desired off-loading but a foot ulcer is not improving, the caregiver might move the patient to a more aggressive care pathway). In another alternative, the data may provide the patient with feedback on the patient's own compliance, optionally providing suggestions to improve compliance with an off-loading protocol.

The data in the orthotic may be accessed by transferring a removable memory from the orthotic to the external device, by wired connection with the orthotic electronics, by wireless communication with the orthotic electronics, or by any combination thereof. The data may be viewed and/or analyzed on a communicating device dedicated for the purpose, on a general purpose computer, on a cell phone, on a wrist-worn data display, on a personal data assistant, etc. In one example, data concerning use of the orthotic may be sent by one protocol (e.g., Bluetooth) to a remote device (e.g., a Bluetooth receiver with a cellular modem at the patient's home) which then sends the data, or a processed form thereof, via a different protocol (e.g., a cell phone protocol) to another remote device (e.g., a physician's office computer, the user's cell phone, etc.). This is exemplary in nature only, and other examples will be readily apparent to those of skill in the art.

In certain embodiments, the present orthotics further comprise a therapeutic for delivery to the foot of the wearer. Preferably, delivery of the therapeutic may be controlled by the computer processor such that delivery of the therapeutic is limited to periods when one or more proximity sensors indicate placement of the orthotic on the wearer's foot. As noted above, it is possible that a proximity sensor may falsely indicate such placement of the orthotic, causing improper delivery of the therapeutic. Thus, in particularly preferred embodiments, the orthotic contains two or more proximity sensors which must be triggered simultaneously before the computer processor initiates delivery of the therapeutic.

In certain embodiments, the therapeutic delivered is low intensity light therapy to tissues of the foot. In preferred embodiments, the orthotic further comprises one or more emitters of low intensity electromagnetic radiation having a peak emission wavelength of between 400 nm and 1200 nm configured for delivery of electromagnetic radiation to the foot of the wearer, and delivery of electromagnetic radiation is controlled by the computer processor such that delivery of is limited to periods when one or more proximity sensors indicate placement of the orthotic on said foot.

In other embodiments, the therapeutic delivered is electrical stimulation of the tissue of the foot. In preferred embodiments, the orthotic further comprises one or more one or more electrodes for delivery of electrical current to the foot of the wearer, and delivery of electrical current is controlled by the computer processor such that delivery of electrical current is limited to periods when one or more proximity sensors indicate placement of the orthotic on said foot.

A preferred footwear orthotic of the present invention comprises a sole portion comprising:
(a) an upper layer for contacting a foot when worn, the upper layer comprising a material having a Shore A of between 30 and 50 to provide cushioning to the foot; and
(b) a rigid or semi-rigid support plate underlying the upper layer which mates with a conforming recess on the bottom of the upper layer.

In these embodiments, it is most preferred that one or more of the proximity sensors, one or more of the pressure sensors, the computer processor, the communications circuit, and the power supply which is operably connected to the proximity sensor(s), pressure sensor(s), computer processor, and communications circuit are housed between this upper layer and support plate.

The term “sole portion” as used herein refers to all or a part of the footwear orthotic which lies underneath the foot when the footwear orthotic is placed on the foot of a wearer for normal use. The sole portion may be further subdivided for purposes of discussion into various subregions including a metatarsal region, an arch region, a heel region, and a midsole region, as depicted in FIG. 1.

The term “rigid” as used herein refers to a support plate which flexes at least 30% less than the upper layer material when exposed to a minimum force which flexes the upper layer material to a 45° angle over a 5 second time interval. In certain embodiments, the support plate flexes at least 50% less than the upper layer material, preferably at least 60% less than the upper layer material, more preferably at least 75% less than the upper layer material, and still more preferably at least 90% less than the upper layer material. The term semi-rigid refers to an otherwise rigid sole plate which comprises one or more “flex lines” in the metatarsal region. These flex lines are engineered locations in the sole plate providing increased flexing during walking relative to the portions of the sole plate lacking such flex lines.

It will be appreciated that the arch region of the orthotic will experience reduced pressure and/or shear forces relative to the metatarsal and heel regions during a normal walking gait in most cases. Thus, in certain embodiments, certain electronic components, most preferably the computer processor and/or battery power supply, are located in the arch region in a recess lying between the upper layer and the sole plate. In preferred embodiments, one or more electronic components in the arch region are further protected from pressure and shear forces by placing a cover between the electronics and the upper layer. For example, a metal or polymeric plate supported by ridges on the support plate may provide a cavity into which the one or more electronic components are placed. In certain embodiments, this cover is supported such that it can withstand at least a 20 psi load, and most preferably at least a 40 psi load, without flexure of the plate causing contact with the electronic components placed underneath. The electronics may further be protected by allowing them to “float” in this cavity, meaning that the electronics are not fastened to the sole plate or cover.

Because the pressure and/or shear forces in the metatarsal and heel regions are somewhat higher, any electronic components placed in this region are also preferably protected by a placing a cover between the electronics and the upper layer. For example a metal or polymeric plate supported by ridges on the sole plate may provide a cavity into which the one or more electronic components are placed. In certain embodiments, this cover is supported such that it can withstand at least an 80 psi load, and most preferably at least a 160 psi load, without flexure of the plate causing contact with the electronic components placed underneath. In preferred embodiments, components in this region are limited to sensors and external connections (e.g., USB or other types of connectors providing access to the computer processor, charging connectors, etc.). Because of the small size of these components relative to the battery and computer processor electronics, higher load forces can be better tolerated.

In order to provide for a certain tolerance to flexing of the orthotic during use, and because electronic components are distributed throughout such a sole portion, flex circuit technology is preferably used to connect the various electronic components. Thus, circuit connections between the electronic components may be made using conductors in or on flexible plastic substrates such as polyimide and PEEK film, or screen printed on polyester substrates.

In order to provide for dissipation of heat within the orthotic during use, and because electronic components generate heat within an enclosure that is sealed, the device optionally includes a design for transmitting heat to the surface of the orthotic. In certain embodiments, this may include a heat conduction material that transfers heat directly to the foot. Thus, the patient's foot will be used as a “heat sink” for dissipating excess heat from the electronics. In other embodiments, a recirculating gas or liquid cooling channel, such as an air channel, may be used to cool the electronics.

In certain embodiments, the upper layer is sealed to the support plate in a liquid-impermeable manner. This is particularly useful in providing an orthotic that is washable in order to reduce contamination of any foot wounds. In these embodiments, the use of inductive charging circuits and/or wireless communications circuits can be particularly advantageous.

In certain embodiments, the upper layer comprises an antimicrobial material. In various embodiments the upper layer may be impregnated with an antimicrobial material and/or may be coated with an antimicrobial material. In the case of a coating, a removable and disposable upper cover may be placed on top of the upper layer, which cover provides the antimicrobial material. Various antimicrobials are known in the art, including silver materials, Cutimed® Sorbact® hydrophobic materials, quaternary ammonioalkyl acrylate polymer hydrogels, etc. This list is not meant to be limiting.

In addition, or in the alternative, the orthotic may be provided with a device which irradiates one or more surfaces of the orthotic with UV light in order to reduce microbial contamination of the orthotic. For example, a stand may be provided which is configured to hold the orthotic when not in use for exposure to UV light. This “UV stand” may be configured to contact the one or more proximity sensors of the orthotic, and/or the stand may itself include one or more proximity sensors. The UV radiation sources may be configured to illuminate only when the desired proximity sensor(s) indicate that the orthotic is properly placed on the stand. Advantageously, such a stand may also provide for recharging of a battery power supply within the orthotic as described herein.

It has been suggested that variations in the micro vascular blood flow and/or onset of inflammation can affect local skin temperature, and that skin temperature variation can be used in the diagnosis and monitoring of micro-circulatory failure and injuries related thereto, including diabetic foot ulcers. Thus, in certain embodiments, the orthotic further comprises one or more temperature sensors configured to generate an electronic signal indicative of a skin temperature on the foot. The temperature sensors are operably connected to the computer processor such that the computer processor receives data indicative of the measured skin temperature(s) and stores that data for future retrieval.

A temperature difference observed between different temperature sensors can be used for predicting the onset of diabetic foot ulcer. Thus, in certain embodiments the temperature data from at least two temperature sensors is analyzed and an increase in temperature is reported to one or more individuals involved in care of the patient. This can include the patient and/or one or more caregivers. The analysis may include comparison of data obtained from spatially distinct locations on the same foot, and/or may include comparison of data obtained from one or more locations on each foot of the individual; for example, the difference in temperature between symmetric locations on each of an individual's feet could be used to determine or predict an injury.

A combination of feedback from sensors can be used for initiating delivery of therapeutic treatment. Thus, in certain embodiments a pre defined pressure signature can be combined with proximity sensing to initiate a therapy ready state, device reset, data collection or other functions. These signatures, which are patient initiated, can be combined with independent forms of patient or physician input to provide more complex or secure instructions.

Other types of sensors may also find use in the orthotics of the present invention, including, but not limited to, one or more of the following:

one or more transcutaneous oxygen sensors (e.g., StO₂, TcpO₂) configured to generate an electronic signal indicative of percent hemoglobin oxygen saturation in tissue, transcutaneous partial pressor of oxygen, etc. of the foot, wherein the transcutaneous oxygen sensor(s) are operably connected to the computer processor such that the computer processor receives data indicative of percent hemoglobin oxygen saturation, transcutaneous partial pressor of oxygen, etc., and stores that data for future retrieval;

one or more accelerometers configured to generate an electronic signal indicative of one or more measures of activity of the wearer, wherein the accelerometer(s) are operably connected to the computer processor such that the computer processor receives data indicative of the one or more measures of activity and stores the data for future retrieval;

one or more moisture or humidity sensors configured to generate an electronic signal indicative of moisture on or adjacent to the foot of the wearer, wherein the moisture or humidity sensor(s) are operably connected to the computer processor such that the computer processor receives data indicative of the moisture on or adjacent to the foot and stores the data for future retrieval;

one or more pH sensors configured to generate an electronic signal indicative of pH on or adjacent to the foot of the wearer, wherein the pH sensor(s) are operably connected to the computer processor such that the computer processor receives data indicative of the pH on or adjacent to the foot and stores the data for future retrieval; and/or

one or more laser doppler sensors configured to generate an electronic signal indicative of vascular blood flow in the foot of the wearer, wherein the laser doppler sensor(s) are operably connected to the computer processor such that the computer processor receives data indicative of the vascular blood flow in the foot and stores the data for future retrieval.

The following is a non-limiting list of preferred embodiments:
1. A footwear orthotic for monitoring delivery of therapy to an injured foot, comprising:
   (i) at least one proximity sensor configured to generate an electronic signal indicative of placement of the orthotic on a foot of a wearer;
   (ii) a computer processor operably connected to said at least one proximity sensor and said at least one pressure sensor, wherein said computer processor receives data indicative of periods of placement of the orthotic on said foot and stores said data for future retrieval;
   (iii) a power supply operably connected to said at least one proximity sensor, said at least one pressure sensor, and said computer processor; and
   (iv) a communications circuit configured to provide communication of data received by said computer processor, or a processed form thereof, to a display or to a second computer processor external to said orthotic.
2. A footwear orthotic according to embodiment 1, further comprising:
   (v) at least one pressure sensor configured to generate an electronic signal indicative of weight-bearing use of the orthotic by said wearer, wherein said computer processor receives data indicative of periods of weight bearing use of the orthotic by said wearer and stores said data for future retrieval.
3. The orthotic of embodiment 2, wherein the at least one pressure sensor generates electronic signals indicative of pressures detected at a plurality of locations within the orthotic.
4. The orthotic of embodiment 3, wherein the electronic signals indicative of pressures detected at a plurality of locations are used to determine a pressure profile, and said pressure profile is used to identify the wearer for initiation of a therapy protocol.
5. The orthotic of any one of embodiments 1 to 4 comprising at least two proximity sensors, wherein said computer processor is configured to store data indicative of placement of the orthotic on the foot of said wearer when said computer processor receives indicative electronic signals from each of said at least two proximity sensors simultaneously.
6. The orthotic of any one of embodiments 1 to 5, further comprising a therapeutic for delivery to the foot of said wearer, wherein delivery of said therapeutic is controlled by said computer processor such that delivery of said therapeutic is limited to periods when said at least one proximity sensor indicates placement of the orthotic on said foot.
7. The orthotic of any one of embodiments 1 to 5, further comprising one or more one or more emitters of low intensity electromagnetic radiation having a peak emission wavelength of between 400 nm and 1200 nm for delivery of electromagnetic radiation to the foot of said wearer, wherein delivery of said electromagnetic radiation is controlled by said computer processor such that delivery of said electromagnetic radiation is limited to periods when said at least one proximity sensor indicates placement of the orthotic on said foot.
8. The orthotic of any one of embodiments 1 to 5, further comprising one or more one or more electrodes for delivery of electrical current to the foot of said wearer, wherein delivery of said electrical current is controlled by said computer processor such that delivery of said electrical current is limited to periods when said at least one proximity sensor indicates placement of the orthotic on said foot.
9. The orthotic of any of embodiments 1-8, wherein said orthotic comprises an insole comprising:
   (a) an upper layer for contacting said foot, said upper layer comprising a material having a Shore A of between 30 and 50 for cushioning said foot; and
   (b) a rigid or semi-rigid support plate underlying said upper layer which mates with a conforming recess on the bottom of said upper layer,
      wherein said at least one proximity sensor, said at least one pressure sensor, said computer processor, said power supply operably connected to said at least one proximity sensor, said at least one pressure sensor, and said computer processor, and said communications circuit are housed between said upper layer and said support plate.
10. The orthotic of embodiment 9, wherein said upper layer is sealed to said support plate in a liquid-impermeable manner.
11. The orthotic of embodiment 10, wherein said orthotic is washable.
12. The orthotic of any of embodiments 1-11, further comprising an inductive charging circuit for recharging said power supply.
13. The orthotic of any of embodiments 1-12, wherein said at least one proximity sensor is overlaid by a protective cover to protect components of said at least one sensor from damage during weight-bearing use of the orthotic by said wearer.
14. The orthotic of any of embodiments 1-13, wherein said orthotic comprises one proximity sensor positioned between the toe and midsole regions of said orthotic and one proximity sensor positioned between the heel and midsole regions of said orthotic.
15. The orthotic of embodiment 14, wherein said proximity sensor positioned between the toe and midsole regions is positioned in the metatarsal region, and said proximity sensor positioned between the heel and midsole regions is positioned in the heel region.
16. The orthotic of any of embodiments 1-15, wherein said orthotic further comprises one or more temperature sensors configured to generate an electronic signal indicative of skin temperature, wherein said one or more temperature sensors are operably connected to said computer processor whereby said computer processor receives data indicative of said skin temperature and stores said data for future retrieval.
17. The orthotic of any of embodiments 1-17, wherein said orthotic further comprises one or more transcutaneoous oxygen sensors configured to generate an electronic signal indicative of percent hemoglobin oxygen saturation in tissue or transcutaneous partial pressure of oxygen, wherein said one or more transcutaneous oxygen sensors are operably connected to said computer processor whereby said computer processor receives data indicative of said of percent hemoglobin oxygen saturation or transcutaneous partial pressure of oxygen and stores said data for future retrieval.
18. The orthotic of any of embodiments 1-17, wherein said orthotic further comprises one or more accelerometers configured to generate an electronic signal indicative of one or more measures of activity of said wearer, wherein said one or more accelerometers are operably connected to said computer processor whereby said computer processor receives data indicative of said one or more measures of activity and stores said data for future retrieval.
19. The orthotic of any of embodiments 1-18, wherein said orthotic further comprises one or more moisture or humidity sensors configured to generate an electronic signal indicative of moisture on or adjacent to the foot of said wearer, wherein said one or more moisture or humidity sensors are operably connected to said computer processor whereby said computer processor receives data indicative of said moisture on or adjacent to the foot and stores said data for future retrieval.
20. The orthotic of any of embodiments 1-19, wherein said orthotic further comprises one or more pH sensors configured to generate an electronic signal indicative of pH on or adjacent to the foot of said wearer, wherein said one or more pH sensors are operably connected to said computer processor whereby said computer processor receives data indicative of said pH on or adjacent to the foot and stores said data for future retrieval.
21. The orthotic of any of embodiments 1-20, wherein said orthotic further comprises one or more laser doppler sensors configured to generate an electronic signal indicative of vascular blood flow in the foot of said wearer, wherein said one or more laser doppler sensors are operably connected to said computer processor whereby said computer processor receives data indicative of said vascular blood flow in the foot and stores said data for future retrieval.
22. The orthotic of any of embodiments 1-21, wherein said orthotic is an insole.
23. The orthotic of any of embodiments 1-21, wherein said orthotic is a walking boot.
24. The orthotic of any of embodiments 1-21, wherein said orthotic comprises an insole comprising an upper layer for contacting said foot, said upper layer comprising a material having a Shore A of between 30 and 50 for cushioning said foot, said upper layer further comprising an antimicrobial material.
25. The orthotic of embodiment 16, wherein said orthotic comprises two or more temperature sensors configured to generate an electronic signal indicative of skin temperature at two or more spatially separated regions of the foot, wherein said temperature sensors are operably connected to said computer processor whereby said computer processor receives data indicative of said skin temperature and determines a difference in temperature between said two or more spatially separated regions.
26. The orthotic of one of embodiments 1-25, wherein the communications circuit provides wireless transmission of data from the orthotic to a computer processor external to the orthotic.
27. The orthotic of one of embodiments 1-25, wherein the orthotic comprises: a shoe which comprises said power supply and said computer processor; and an insole which comprises said proximity sensor, wherein said insole receives energy from said power supply inductively and said computer processor receives data from said proximity sensor inductively.
28. The orthotic of embodiment 27, wherein the insole comprises a battery which is inductively charged by said power supply.
29. The orthotic of any of embodiments 1-28, further comprising a user input device, wherein a signal from said user input device is used by the computer processor to determine compliance with use of the orthotic by the wearer.
30. The orthotic of embodiment 29, wherein the user input device detects a biometric signal indicative of the desired user.
31. The orthotic of embodiment 29, wherein receipt of a predetermined signal from the user input device by the computer processor is used to initiate a treatment regimen.
32. The orthotic of embodiment 3, wherein the electronic signals indicative of pressures detected at a plurality of locations are used to determine if movement of the wearer's foot and/or weight bearing use of the orthotic remain within or exceed predetermined parameters.
33. The orthotic of any of embodiments 1-32, wherein sensor signals from the orthotic are used to determine compliance with a therapy regimen.
34. The orthotic of embodiments 33, wherein the therapy regimen comprises predetermined periods of use of the orthotic.
35. A method, comprising placing an orthotic of any of embodiments 1-34 on the foot of a wearer.

It is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

The present invention and the various features and advantageous details thereof are explained more fully with reference to the non-limiting embodiments that are illustrated in the accompanying drawings and detailed in the following description. It should be noted that the features illustrated in the drawings are not necessarily drawn to scale. Descriptions of well-known components and processing techniques are omitted so as to not unnecessarily obscure the present invention. The examples used herein are intended merely to facilitate an understanding of ways in which the invention may be practiced and to further enable those of skill in the art to practice the invention. Accordingly, the examples should not be construed as limiting the scope of the invention. In the drawings, like reference numerals designate corresponding parts throughout the several views.

FIG. 1 depicts a schematic diagram of an exemplary orthotic.

FIG. 2 depicts a schematic diagram of a sole plate of an exemplary orthotic.

### DETAILED DESCRIPTION

The etiology of diabetic foot ulcerations is commonly associated with the presence of peripheral neuropathy and repetitive trauma due to normal walking activities to areas of the foot exposed to moderate or high pressure. The goal of treatment plans generally include as a central tenet the mitigation or modulation of the activity and/or pressure which initiated the injury. While numerous studies have detailed the potential pressure off-loading properties of various treatment modalities, studies have also suggested that, if easily removable, these therapies will likely not be used for the majority of steps taken each day. See, e.g., Wu and Armstrong, Plast. Reconstr. Surg. 2006 June;117(7 Suppl):248S-253S; Crews et al., J. Am. Podiatr. Med. Assoc. 2009 March-April;99(2):100-3.

A. Orthotic Materials

The purpose of the orthotic is to provide the necessary support for the various flexion positions of the foot. Forces in the foot change dramatically during the various phases of a person's gait. For example, at heel strike an entire individual's weight is being applied at the heel of the foot. At this stage the purpose of the inner sole is to cup the heel. At mid-stance, the individual's weight is spread out more evenly across the foot and the inner sole must provide adequate support to the arch of the foot. During toe-off, the individual's weight is concentrated at the balls of the feet and the insole must be able to flex and stabilize the foot. The orthotic is preferably flexible in order to maintain contact with the foot during gait movements.

In the case of the present orthotic, the orthotic may be formed by lamination of an upper conforming top material having a hardness of 40 shore 00 to 45 shore A, to a lower shell material having a hardness of 70 shore A to 80 shore D which provides support for the foot and shielding for the electronics from damage. To the shell material, glass can be added up to about 30% to increase stiffness if needed. Suitable materials for the conforming material include appropriate durometer Plastazote (a heat moldable polyethylene foam material). Alternative materials in addition to Plastazote include Dynafoam (a polyvinyl chloride foam), Ortho felt (a resilient blend of cotton and wool), Spenco (a neoprene sponge covered with multistretch nylon), Molo (a combination of latex, jells, leather, and cork incorporated into a rubbery sheet) and, PPT (an open cell, porous, firm foam material). The insert layer of Plastazote material ranges in thickness from ⅛ to ¼ inch and the shell layer ranges in thickness from ¼ to ½ inch depending on the arch height, heel shape and other factors, including the need to accommodate and protect the electronics.

The upper surface may be scored into small hexagon or other shape of roughly ⅜ inches across or smaller, and one or more of the hexagonal or other shape areas directly under an ulceration or pressure site can be removed to create a reduction in pressure at the ulcer site. If desired, the resulting hole in the insert material may be partially filled with a 15 durometer polyurethane fill material (e.g., Poron) that is softer than the insert material. This fill material may be pre-shaped to be accommodated into the pre-scored hexagonal or other shape areas. In the case where the area to be removed contains an optical source, the optical source may be moved to an adjacent area, or inserted into the fill material. In such a case, the fill material or the insert may comprise pre-existing locations into which the optical source may be placed.

A custom orthotic fabrication process may be used to improve the fit of the orthotic. Such a fabrication incorporates forming an impression of the patient's foot. A foam impression of the patient's foot may be made using a 14 inch foam box. The foot is then held in a neutral position by grasping just below the ankle bone with the technician's thumb and index finger on one hand. At the same time with the other hand apply 2 or 3 fingers on the first metatarsal. While holding the patient's foot in this position, the patient applies downward pressure on the foam material until they meet resistance, the ankle and first metatarsal are held firmly as the impression is being made to avoid tilting of the foot. After the impression has been made and before removing the foot from the foam, the technician firmly pushes down the ends of the toes so that they are not elevated (dorsiflexed). The foot is then removed from the foam.

Once this is completed, the fabrication process begins by pouring liquid plaster of paris into the impression and waiting for it to harden. Once hardened, the cast is sanded smooth in a manner that is consistent with standard orthotic lab procedures for the fabrication of an accommodated orthotic. This orthotic material, which is provided in sheets with the layers laminated together, is cut to a size that is slightly larger than the foot and placed in a convection oven at 250° F. for 2 to 3 minutes until soft. Then the material is placed over the cast which is lying inside a vacuum forming machine with the bottom of the cast (bottom of foot) facing upwards. The vacuum forming machine is closed and the heated material is pulled down over the cast as the air is removed from the vacuum forming chamber to thereby shape the insert material in step 18. The insert is then ground to fit the shape and contour of the shoe and foot. The custom orthotic is then added to or coupled with a shoe and dispensed to the user.

In an alternative to the custom fabrication process described above, a digitizer comprising a set of pins that contact the plantar surface of the foot may be used to provide an image of the foot from which a custom orthotic is constructed. Computerized milling can then be used to convert this image into the form-fitting insert material. Alternative methods of digitization of the foot may be employed, such as digital image capture of the foot surface.

It is often advantageous to maintain a moist environment to promote healing of lower extremity ulcers. The surface of the orthotic that contacts skin is preferably sealed such that the moist environment does not contaminate the electronics carried by the orthotic, and such that exudates or other materials may be easily removed from the orthotic. A silicone or other barrier surface which may be made sufficiently transparent to therapeutic light if necessary may be employed over the upper surface. This barrier surface may be held in place by an adhesive such that it is easily separated from the insert material, thereby providing a replaceable barrier.

The barrier surface can also advantageously provide a replaceable absorptive dressing which will absorb wound exudates, yet maintain a physiologically moist interface between the wound itself and the dressing material. Dressing types include hydrogels/hydrocolloids, alginate dressings, collagen wound dressings, antimicrobial dressings, and synthetic skin substitutes. Suitable dressing materials such as calcium alginate or a hydrogel material can be provided overlying an impermeable barrier surface to protect the electronics of the orthotic.

In an alternative embodiment, the dressing material may be provided separately from the barrier surface in the form of a dressing which is worn like a bandage or a stocking. As in the case of the barrier surface, the portion of the bandage/stocking which lies between the orthotic and the skin surface must be sufficiently transparent to the therapeutic light being generated. The layer most proximal to the skin may provide a substrate on which hydrogels/hydrocolloids, alginate dressings, collagen wound dressings, antimicrobial dressings, and synthetic skin substitutes may be emplaced. While the frequency of dressing change depends on the nature of the wound, the amount of exudate, etc., it is usually performed between two times daily to every other day.

In the case where the orthotic is used to deliver a therapeutic (e.g., low intensity light therapy, electrical stimulation, etc.) to tissues of the foot, it may be advantageous to provide for alignment of the therapeutic to the wound. This alignment may take place at the level of orthotic manufacture, in which case the therapeutic unit may be prepositioned in the orthotic according to data previously acquired. Alternatively, the caregiver or user may perform this alignment. A mark on the orthotic may indicate the positions of therapeutic unit within the orthotic may be indicated by reference markers, which may be aligned with a wound, with the source of pain, etc.

B. Electronics

1. Proximity Sensors

A capacitive proximity sensor essentially comprises an oscillator in which a capacitor is formed by two electrodes placed in front of the sensor. The sensing surface of a capacitive sensor is formed by two concentrically shaped metal electrodes of an unwound capacitor. When an object nears the sensing surface it enters the electrostatic field of the electrodes and changes the capacitance in an oscillator circuit. As a result, the oscillator begins oscillating. The trigger circuit reads the oscillator's amplitude and when it reaches a specific level the output state of the sensor changes. As the target moves away from the sensor the oscillator's amplitude decreases, switching the sensor output back to its original state. No physical contact with the object to be detected is required, and typically detection is irrespective of material or conductivity. Capacitive sensors are commercially available with detection ranges from 1 mm to 50 mm, and include some with adjustable detection distance.

Ultrasonic proximity sensors use a transducer to send and receive high frequency sound signals. When a target enters the beam the sound is reflected back to the switch, causing it to energize or deenergize the output circuit. Piezoelectric Disk A piezoelectric ceramic disk is mounted in the sensor surface. It can transmit and receive high-frequency pulses. A highfrequency voltage is applied to the disk, causing it to vibrate at the same frequency. The vibrating disk produces high-frequency sound waves. When transmitted pulses strike a sound-reflecting object, echoes are produced. The duration of the reflected pulse is evaluated at the transducer. When the target enters the preset operating range, the output of the switch changes state. When the target leaves the preset operating range, the output returns to its original state.

Inductive proximity sensors incorporate an electromagnetic coil which is used to detect the presence of a conductive metal object. The sensor will ignore the presence of an object if it is not metal. This type of sensor consists of four elements: coil, oscillator, trigger circuit, and an output. The oscillator is an inductive capacitive tuned circuit that creates a radio frequency. The electromagnetic field produced by the oscillator is emitted from the coil away from the face of the sensor. The circuit has just enough feedback from the field to keep the oscillator going. When a metal target enters the field, eddy currents circulate within the target. This causes a load on the sensor, decreasing the amplitude of the electromagnetic field. As the target approaches the sensor the eddy currents increase, increasing the load on the oscillator and further decreasing the amplitude of the field. The trigger circuit monitors the oscillator's amplitude and at a predetermined level switches the output state of the sensor from its normal condition (on or off). As the target moves away from the sensor, the oscillator's amplitude increases. At a predetermined level the trigger switches the output state of the sensor back to its normal condition (on or off). Inductive proximity sensors can be used in conjunction with a sock worn on the foot which comprises a metallic indicator for triggering the sensor. Such proximity detection can be less prone to false signals than other types of proximity sensors, particularly when the orthotic is provided with more than one such sensor which must be triggered simultaneously.

2. Pressure Sensors

A number of suitable pressure sensors are known in the art. For example, U.S. Pat. No. 5,373,651 discloses instrumented shoes comprising a plurality of pressure sensors, a microprocessor, a memory and an inductive interface. The microprocessor receives data related to the force exerted upon the shoe from the pressure sensor, stores that data in memory, and transmits the stored data to a remote computer via the inductive interface.

Similarly, U.S. Pat. No. 5,642,096 discloses a footwear article comprising at least one hydrocell carried in an insole. This hydrocell supports a sensor in the liquid mass of the hydrocell which detects both a pressure condition and a temperature condition present in the hydrocell. And U.S. Pat. No. 7,426,873 discloses a shoe having a plurality of sealed cavities contained within the sole thereof, and a plurality of micro electro-mechanical system (MEMS) pressure sensors contained within the sealed cavities.

3. Other Sensors

Variation in the micro vascular blood flow affects local skin temperature and hence skin temperature variation can be used in the diagnosis of micro-circulatory failure. Temperature measurement sensors integrated into the orthotic can be used to measure average temperatures at various locations on the lower extremity, and a temperature difference of >2.2° C. used for predicting the onset of diabetic foot ulcer or monitoring therapy success. Similarly, trancutaneous oxygen tension (Tc_{O2}) sensors, transcutaneous partial pressure of oxygen (TcpO₂) sensors and the like also may be helpful in assessment of the patient. Measurements are usually obtained from several sites on the foot. A Tc_{O2} level of less than 30 mmHg can be used for predicting the onset of diabetic foot ulcer or monitoring therapy success Likewise, pressure and shear patterns may be measured on the plantar surface by means of pressure sensors for predicting the onset of diabetic foot ulcer. One or more such sensors may be incorporated into the present orthotics, or provided on a sensor orthotic which is separate from the light therapy orthotic. When the onset of a lower extremity ulcer is sensed, low level light therapy may be initiated. In addition, the patient or caregiver may be notified by the sensor and its associated electronics.

WO08/058051 discloses a smart insole which comprises a plurality of temperature sensors; an algorithm which compares the data from the temperature sensors to a signature profile, and provides a feedback value; means for communicating the feedback value; and a power source. In other embodiments, this publication discloses a plurality of temperature sensors which generate a signal; a circuit means electrically connected to the plurality of temperature sensors whereby said signal is collected; a transmission means to transmit the signal; a power source electrically connected to said plurality of temperature sensors, circuit means, and transmission means; a software program that receives the transmitted signal and compares the transmitted signal to a signature profile and generates a feedback signal; a feedback means which transmits the feedback signal. Signals are collected from one or more temperature sensors located in sensing proximity to a patient's foot to generate a test profile, and this profile is compared to a signature profile.

U.S. Pat. No. 7,457,724 discloses a shoe which comprises at least one accelerometer for generating acceleration signals, and a processor within the shoe to process the acceleration signals to determine at least one of the speed and distance traveled of a person wearing the shoe. A wireless transmitter configured within the shoe transmits this information to a wireless receiver worn or operated by the person.

Morley et al., IEEE Trans Biomed Eng. 48:815-20, 2001, entitled “In-shoe multisensory data acquisition system,” reports on an electronic system in a shoe that monitors temperature, pressure, and humidity, storing the data in a battery-powered device for later uploading to a host computer for data analysis. The pressure sensors are located at the heel, and under three metatarsal heads. Temperature sensors are located under the medial metatarsal head and under the heel. The humidity sensor is located in the toe of the shoe.

In certain embodiments, the orthotic may be composed of layers of sensors, which may be laminated together in a desired sequence to provide the desired combination of sensors. This may allow for customization of the sensor array according to the needs of the wearer.

3. Battery Modules

Numerous battery technologies are known in the art, including common alkaline batteries, oxyride batteries, lithium batteries, etc. There are three preferred battery technologies that could be employed: Nickel Cadmium (NiCad), Nickel Metal Hydride (NiMH) and Lithium Ion (Li-ion), and most preferred are Li-ion batteries.

4. Recharging

In the case of rechargeable batteries, the battery could be provided with a wired plug in to a conventional charger, or could be provided with an inductive coupling using an inductive coil that would be located on the orthotic. The inductive circuit would be complete upon placing the orthotic or the battery-containing module in a cradle or dock that has a mating inductive coil. Inductive charging is particularly advantageous in the case of an orthotic which is sealed so as to be washable and/or sterilizable, as this would eliminate the need for a port for receiving a charging cord. In addition, regenerative charging of a battery power supply, which converts the kinetic energy of body movement into electrical energy for battery storage, can be used to reduce the size of the battery and to extend wear periods between recharges. This is particularly attractive in the case of orthotics which deliver low level light or electric current as a therapeutic, as this places additional demand on available battery technology.

In order to maintain the battery properly charged, the orthotic may use communications circuitry to signal either proper charge or inadequate charge. For example, an LED may illuminate to indicate a particular charge state. In one alternative, the orthotic processor may store a fault code when the charge falls to an inadequate level, followed by shutting down of the electronics. Upon data access, the caregiver or user can determine that the orthotic was only properly charged at a certain interval. This can provide additional feedback on proper use of the orthotic. In another alternative, the orthotic may communicate with the user to indicate that the charge state of the battery is inadequate. For example, a signal communicated from the orthotic may initiate sending of a message to the user (e.g., a text message to a cellular phone) which instructs the user to begin charging. If this signal is not responded to, a wireless signal from the orthotic may initiate sending of a message to the caregiver warning that the user is not in compliance with the use of the device.

In another alternative, a signal communicated from the orthotic may be received by a resolution center, which initiates action by the resolution center such as tracking the issue (e.g., a low battery state) signaled by the orthotic until it is resolved. The resolution center may contact the user and/or caregiver in an effort to seek action to resolve the issue.

In yet another alternative, a resolution center may periodically receive a signal from the orthotic indicating that the orthotic is able to communicate and that there are no issues with the orthotic. Action by the resolution center may be initiated by a loss of that periodic signal, such as when the battery has inadequate charge. The resolution center may contact the user and/or caregiver in an effort to seek action to resolve the issue.

5. Communications

Data import and export from the sensors may be by wired and/or wireless means. The term “wired” in this context refers to any method in which there is a physical contact which operably connects the control module to external display or processing device, such as a PDA, computer, cellular telephone, network connection, display, etc., which displays data from, sends data to, or retrieves data from the control module. The term “wireless” refers to any method in which data is sent to or retrieved from the control module without a physical connection.

In the case of a wired data transfer, a cabled USB connection between the control module and the external device is one example that may be provided. Alternatively, a memory card, such as a Memory Stick, Secure Digital, Flash memory drive, etc., may be used to transfer data by moving the memory card between the electronic module of the orthotic and the external device. The orthotic may also comprise a display such as a small LED similar to an iPod, cell phone, or watch screen for displaying data. This screen may be part of the orthotic, or may reversibly attach to the orthotic for display as desired. In the case where a display is part of the orthotic, it may be advantageous to activate the display only when necessary in order to conserve battery life.

In the case of a wireless data transfer, numerous standards well known in the art may be used. Such wireless connections include various radio frequency and optical (e.g., infrared) connections that are known in the art. For relatively short distance RF communications, Bluetooth, HomeRF, IEEE 802.11b, IEEE 802.11a, and IEEE 802.15.4 are well known standard communications protocols that may be used. For somewhat longer range data transfers, cellular telephone protocols such as CDMA, TDMA, GSM, and WAP may be employed.

These methods need not be used in isolation, but instead may be advantageously employed in combination. For example, the electronic module of the orthotic may communicate at a short distance with a local “base station” by a wired or wireless mechanism, and the base station may then communicate with an external device, for example at a caregiver's office or central data collection point, using one of the cellular telephone protocols, or through telephone twisted pair, cable TV, or other wiring existing in the user's location. This can extend battery life in the orthotic by lowering power requirements for communication, while the base station may be powered by line voltage.

6. Processors

Suitable processor systems are readily available commercially. A suitable processor can comprise analog to digital conversion of the sensor signals, a microprocessor, memory for storage of programming and/or acquired data, and interfacing circuitry. In embedded systems of this type, the software typically resides in firmware, such as a flash memory or read-only memory (ROM) chip, in contrast to a general-purpose computer that loads its programs into random access memory (RAM) each time.

C. Exemplary Orthotic

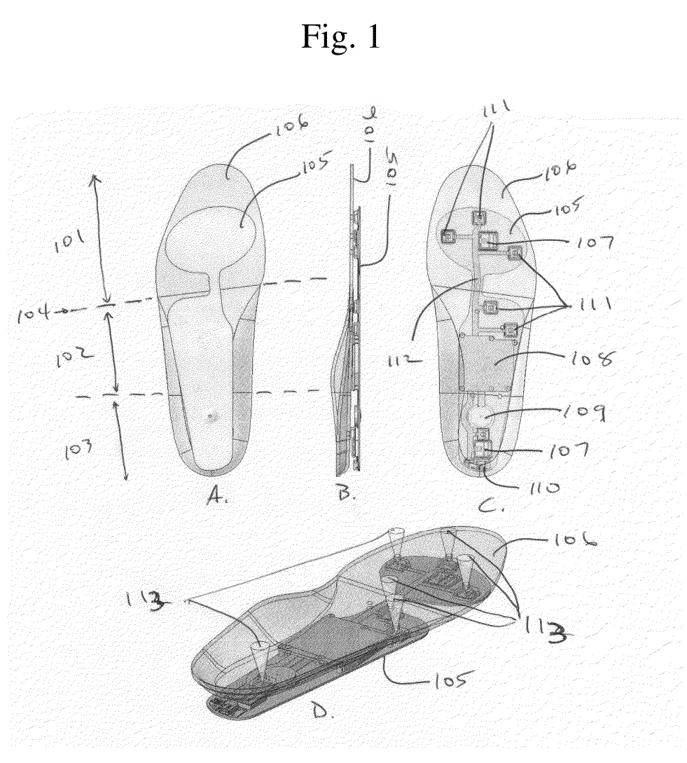

FIG. 1 depicts a preferred embodiment of an orthotic of the present invention in bottom (A), side (B), and transparent (C and D) views. While depicted as an insole structure, the combination of elements are equally applicable to use in a shoe sole or other orthotic structure. In such an embodiment, the footwear orthotic may include a shoe or boot comprising some or all of the electronic components described herein, and the orthotic may comprise a disposable insole material for use inside the shoe or boot which does not carry any of the electronics or which carries only a subset of electronics. For example, the shoe sole may contain the processor circuitry and battery, and the insole may contain a (comparatively) smaller battery and one or more proximity sensors. The insole electronics may be inductively coupled to the electronics (i.e., the battery in the sole may be used to inductively charge the battery or power the electronics in the insole; the sensor(s) in the insole may communicate inductively with the processor in the sole; etc.), may connect by means of “contacts” on the insole and shoe or boot, or by a combination thereof. The insole may provide a conforming top material having a hardness of 40 shore 00 to 45 shore A, while the shoe or boot may provide a rigid underlayer material having a hardness of 70 shore A to 80 shore D.

The orthotic can be divided into several regions based on the portion of the foot which is intended to contact the orthotic. These are the metatarsal region 101 (running from a midsole position 104 to the toe), the arch region 102, and the heel region 103. A rigid or semi rigid bottom support plate 105 is mated to a conforming upper layer 106. While not depicted in this figure, the support plate 105 is preferably designed to fit into a matching recess in the bottom of upper layer 106 and sealed at the periphery, for example by gluing. The arch region 108 contains a recess into which a computer processor, battery, and associated circuitry is installed, protected by a rigid cover plate 108 or other protective covering. For example, the electronics may be embedded in a polymeric material which is poured into a mold and allowed to cure to form a shell around the electronic components. A pair of proximity sensors 107 are provided in the metatarsal and heel regions, and a pressure sensor 109 is provided in the heel region. A USB-type data port 110 is provided at the heel, which can also provide for wired recharging of the battery. The electronics are connected via appropriate flex circuitry 112.

The depicted embodiment also provides emitters for delivering low intensity phototherapy to the foot as described generally in, for example, U.S. Pat. No. 6,454,791. The circuit includes an array of radiation emitters 111 (e.g., lasers) electrically connected in series. The plurality of emitters may be encapsulated, for example in an optically clear epoxy material, to maintain the relative position of the emitters, present a low profile for the circuitry, and to protect the emitters from contamination by debris. The emitters are preferably lasers such as a vertical-cavity surface emitter (VCSEL) having a peak emission wavelength on the order of 400-1300 nm, with the preferred power output at least 5-10 mw per VCSEL and the preferred wavelength being between 760 to 850 nanometers. As an example, Philips Technologie GmbH U-L-M Photonics manufactures VCSELs having emission wavelengths between approximately 760 nm and 1000 nm, such as ULM 850-01-TT-HSMDCA which emits light at a peak wavelength of 850 nm. Emission from radiation emitters 111 are depicted as cones 113 in FIG. 1. Various forms of medical treatment using lasers and VCSELs are disclosed in U.S. Pat. No. 5,616,140. Other types of emitters, such as light emitting diodes, can be used together with, or instead of, laser emitters.

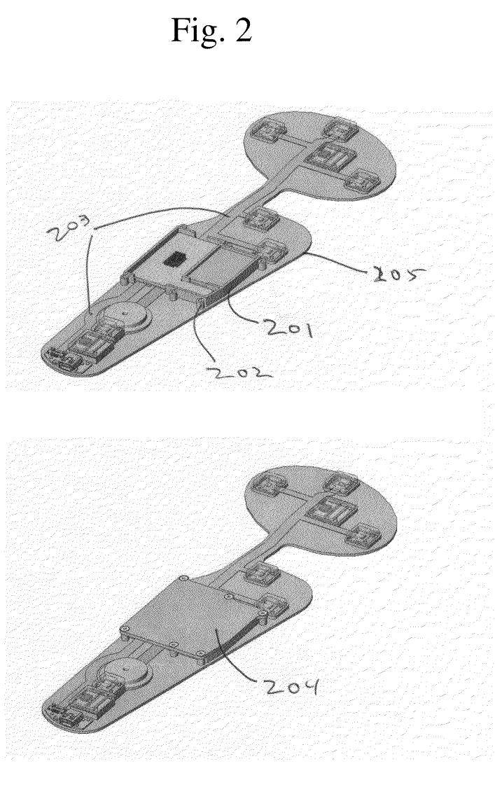

FIG. 2 depicts details of an exemplary support plate 205. Ridges on the support plate 201 form enclosures for protection of the various electronic components. In addition, posts 202 provide attachment positions for protective plate(s) 204 overlying some or all of the components. The electronics are preferably formed using flex circuit technology and connected by connectors 203.

Preferred components can include lithium ion polymer batteries having a rated capacity of about 180 mAh at a nominal voltage of 3.7V. Such cells can have a weight of about 4.5 g and dimensions of about 20×30×4 mm. Proximity sensors can be QT100A charge-transfer (‘QT’) touch sensor (Quantum Research Group), which provides a self-contained digital IC package having a settable sensitivity. The power supply for such a device can range between 2.0V and 5.5V. The processor can be a PIC16F882/883/884/886/887 family microcontroller (Microchip Technology), which includes an on-circuit high-endurance Flash/EEPROM cell, serial communications, and A/D conversion. The pressure sensor can be a Force Sensing Resistor (FSR) such as the FSR-400 (Interlink Electronics), which is a polymer thick film (PTF) device which exhibits a decrease in resistance with an increase in the force applied to the active surface. This list of materials is exemplary in nature only.

While the invention has been described and exemplified in sufficient detail for those skilled in this art to make and use it, various alternatives, modifications, and improvements should be apparent without departing from the spirit and scope of the invention. The examples provided herein are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of and “consisting of may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

What is claimed:

1. A footwear orthotic for monitoring delivery of therapy to an injured foot, comprising:
(i) at least one proximity sensor configured to generate an electronic signal indicative of placement of the orthotic on a foot of a wearer;
(ii) a computer processor operably connected to said at least one proximity sensor and said at least one pressure sensor, wherein said computer processor receives data indicative of periods of placement of the orthotic on said foot and stores said data for future retrieval;
(iii) a power supply operably connected to said at least one proximity sensor, said at least one pressure sensor, and said computer processor; and
(iv) a communications circuit configured to provide communication of data received by said computer processor, or a processed form thereof, to a display or to a second computer processor external to said orthotic.

2. A footwear orthotic according to claim 1, further comprising:
(v) at least one pressure sensor configured to generate an electronic signal indicative of weight-bearing use of the orthotic by said wearer, wherein said computer processor receives data indicative of periods of weight bearing use of the orthotic by said wearer and stores said data for future retrieval.

3. The orthotic of claim 2, wherein the at least one pressure sensor generates electronic signals indicative of pressures detected at a plurality of locations within the orthotic.

4. The orthotic of claim 3, wherein the electronic signals indicative of pressures detected at a plurality of locations are used to determine a pressure profile, and said pressure profile is used to identify the wearer for initiation of a therapy protocol.

5. The orthotic of claim 1, comprising at least two proximity sensors, wherein said computer processor is configured to store data indicative of placement of the orthotic on the foot of said wearer when said computer processor receives indicative electronic signals from each of said at least two proximity sensors simultaneously.

6. The orthotic claim 1, further comprising a therapeutic for delivery to the foot of said wearer, wherein delivery of said therapeutic is controlled by said computer processor such that delivery of said therapeutic is limited to periods when said at least one proximity sensor indicates placement of the orthotic on said foot.

7. The orthotic of claim 1, further comprising one or more one or more emitters of low intensity electromagnetic radiation having a peak emission wavelength of between 400 nm and 1200 nm for delivery of electromagnetic radiation to the foot of said wearer, wherein delivery of said electromagnetic radiation is controlled by said computer processor such that delivery of said electromagnetic radiation is limited to periods when said at least one proximity sensor indicates placement of the orthotic on said foot.

8. The orthotic of claim 1, further comprising one or more one or more electrodes for delivery of electrical current to the foot of said wearer, wherein delivery of said electrical current is controlled by said computer processor such that delivery of said electrical current is limited to periods when said at least one proximity sensor indicates placement of the orthotic on said foot.

9. The orthotic of claim 1, wherein said orthotic comprises an insole comprising:
(a) an upper layer for contacting said foot, said upper layer comprising a material having a Shore A of between 30 and 50 for cushioning said foot; and
(b) a rigid or semi-rigid support plate underlying said upper layer which mates with a conforming recess on the bottom of said upper layer,
wherein said at least one proximity sensor, said at least one pressure sensor, said computer processor, said power supply operably connected to said at least one proximity sensor, said at least one pressure sensor, and said computer processor, and said communications circuit are housed between said upper layer and said support plate.

10. The orthotic of claim 9, wherein said upper layer is sealed to said support plate in a liquid-impermeable manner.

11. The orthotic of claim 10, wherein said orthotic is washable.

12. The orthotic of claim 1, further comprising an inductive charging circuit for recharging said power supply.

13. The orthotic of claim 1, wherein said at least one proximity sensor is overlaid by a protective cover to protect components of said at least one sensor from damage during weight-bearing use of the orthotic by said wearer.

14. The orthotic of claim 1, wherein said orthotic comprises one proximity sensor positioned between the toe and midsole regions of said orthotic and one proximity sensor positioned between the heel and midsole regions of said orthotic.

15. The orthotic of claim 14, wherein said proximity sensor positioned between the toe and midsole regions is positioned in the metatarsal region, and said proximity sensor positioned between the heel and midsole regions is positioned in the heel region.

16. The orthotic of claim 1, wherein said orthotic further comprises one or more temperature sensors configured to generate an electronic signal indicative of skin temperature, wherein said one or more temperature sensors are operably connected to said computer processor whereby said computer processor receives data indicative of said skin temperature and stores said data for future retrieval.

17. The orthotic of claim 1, wherein said orthotic further comprises one or more transcutaneoous oxygen sensors configured to generate an electronic signal indicative of percent hemoglobin oxygen saturation in tissue or transcutaneous partial pressure of oxygen, wherein said one or more transcutaneous oxygen sensors are operably connected to said computer processor whereby said computer processor receives data indicative of said of percent hemoglobin oxygen saturation or transcutaneous partial pressure of oxygen and stores said data for future retrieval.

18. The orthotic of claim 1, wherein said orthotic further comprises one or more accelerometers configured to generate an electronic signal indicative of one or more measures of activity of said wearer, wherein said one or more accelerometers are operably connected to said computer processor whereby said computer processor receives data indicative of said one or more measures of activity and stores said data for future retrieval.

19. The orthotic of claim 1, wherein said orthotic further comprises one or more moisture or humidity sensors configured to generate an electronic signal indicative of moisture on or adjacent to the foot of said wearer, wherein said one or more moisture or humidity sensors are operably connected to said computer processor whereby said computer processor receives data indicative of said moisture on or adjacent to the foot and stores said data for future retrieval.

20. The orthotic of claim 1, wherein said orthotic further comprises one or more pH sensors configured to generate an electronic signal indicative of pH on or adjacent to the foot of said wearer, wherein said one or more pH sensors are operably connected to said computer processor whereby said computer processor receives data indicative of said pH on or adjacent to the foot and stores said data for future retrieval.

21. The orthotic of claim 1, wherein said orthotic further comprises one or more laser doppler sensors configured to generate an electronic signal indicative of vascular blood flow in the foot of said wearer, wherein said one or more laser doppler sensors are operably connected to said computer processor whereby said computer processor receives data indicative of said vascular blood flow in the foot and stores said data for future retrieval.

22. The orthotic of claim 1, wherein said orthotic is an insole.

23. The orthotic of claim 1, wherein said orthotic is a walking boot.

24. The orthotic of claim 1, wherein said orthotic comprises an insole comprising an upper layer for contacting said foot, said upper layer comprising a material having a Shore A of between 30 and 50 for cushioning said foot, said upper layer further comprising an antimicrobial material.

25. The orthotic of claim 16, wherein said orthotic comprises two or more temperature sensors configured to generate an electronic signal indicative of skin temperature at two or more spatially separated regions of the foot, wherein said temperature sensors are operably connected to said computer processor whereby said computer processor receives data indicative of said skin temperature and determines a difference in temperature between said two or more spatially separated regions.

26. The orthotic of claim 1, wherein the communications circuit provides wireless transmission of data from the orthotic to a computer processor external to the orthotic.

27. The orthotic of claim 1, wherein the orthotic comprises:
a shoe which comprises said power supply and said computer processor; and
an insole which comprises said proximity sensor, wherein said insole receives energy from said power supply inductively and said computer processor receives data from said proximity sensor inductively.

28. The orthotic of claim 27, wherein the insole comprises a battery which is inductively charged by said power supply.

29. The orthotic of claim 1, further comprising a user input device, wherein a signal from said user input device is used by the computer processor to determine compliance with use of the orthotic by the wearer.

30. The orthotic of claim 29, wherein the user input device detects a biometric signal indicative of the desired user.

31. The orthotic of claim 29, wherein receipt of a predetermined signal from the user input device by the computer processor is used to initiate a treatment regimen.

32. The orthotic of claim 3, wherein the electronic signals indicative of pressures detected at a plurality of locations are used to determine if movement of the wearer's foot and/or weight bearing use of the orthotic remain within or exceed predetermined parameters.

33. The orthotic of claim 1, wherein sensor signals from the orthotic are used to determine compliance with a therapy regimen.

34. The orthotic of claim 33, wherein the therapy regimen comprises predetermined periods of use of the orthotic.
